(19) United States
(12) Patent Application Publication
(10) Pub. No.: US 2021/0267650 A1
(43) Pub. Date: Sep. 2, 2021

(54) SCREW CADDY

(71) Applicant: ABLE MEDICAL DEVICES, Marquette, MI (US)

(72) Inventors: Peter J. Didyk, Northville, MI (US); Joseph Mohar, Marquette, MI (US)

(73) Assignee: J.M. Longyear Manufacturing, LLC dba Able Medical Devices, Marquette, MI (US)

(21) Appl. No.: 16/802,713

(22) Filed: Feb. 27, 2020

(51) Int. Cl. A61B 17/80; A61B 17/68; A61B 17/86; A61B 50/20

(52) U.S. Cl. CPC A61B 17/808; A61B 17/686; A61B 2017/90; A61B 50/20; A61B 17/866

(57) ABSTRACT

A fastener caddy for use with a bone plate. The fastener caddy includes preloaded fasteners that are positioned above the fastener apertures in the bone plate once the caddy is coupled to the bone plate. The caddy permits the driving of fasteners with the fastener already in proper alignment and without the need to retrieve each individual fastener. Once the fasteners are installed into the bone plate, the caddy is removed using an insertion tool.

### BACKGROUND OF THE INVENTION

The present invention generally relates to surgical equipment and, more particularly, to surgical instruments used to fix or locate segments of bone.

Bone plates and surgical fasteners, such as screws, are used for the fixation of bone during surgery. A standard bone plate is a piece of material, usually metal, having circular and/or slotted holes through which bone screws are fixated. Typically, the bone plate is used to span a discontinuity between two adjacent bones or bone segments, and the screws are placed through holes in the bone plate that are positioned on both sides of the fracture to secure the plate to the bone segments to promote fusion between the adjacent bone or bone segments.

The length, shape, contour, and segments of a bone plate can vary, depending upon where the bone plate is inserted. For example, some bone plates could have multiple segments, while others could have a single segment. Moreover, multiple segment bone plates can include spaced apart segments.

Typically once the bone plate and fasteners (anchors) are sterilized, the surgeon will locate the bone plate on the bone and then insert each individual fastener, one at a time. This requires another person to provide the screws to the surgeon and/or the surgeon to retrieve a screw from a screw rack. This is a time-consuming process. Moreover, the surgeon will need to locate each individual screw over each individual hole in the bone plate. Again, this takes additional time and can also result in screws becoming cross-threaded and/or being advanced too far or not far enough into the bone.

Accordingly, the need exists for a surgical device that pre-locates the screws over the holes in the bone plate and permits the screws to be easily advanced by use of a driving tool, while maintaining the screws in the proper alignment and preventing over-tightening of the screws.

### SUMMARY OF THE INVENTION

One aspect of the present invention is a surgical plate and cavity assembly. The assembly includes a bone plate having a top surface, a bottom surface, and a plurality of fastener apertures. The surgical plate and caddy assembly also includes a caddy that is coupled to the bone plate. The caddy has a top surface, a bottom surface, and at least two side surfaces. The caddy also has an inserter aperture and a plurality of fastener apertures that are aligned with the plurality of fastener apertures in the bone plate when the caddy is coupled to the bone plate. At least two of the caddy side surfaces include a clip portion that has a tab that extends below and contacts the bottom surface of the bone plate. At least one of the clip portions will deflect to remove the tab from contact with the bottom surface of the bone plate when the insertion tool is inserted into the inserter aperture. The surgical plate and caddy assembly also includes a plurality of fasteners that are inserted into the plurality of fastener apertures in the caddy.

Another aspect of the present invention is a method of installing fasteners into the bone plate. The method includes forming a caddy that has a top surface, a bottom surface, and at least two clip portions. The caddy also has at least one insertion aperture and a plurality of fastener apertures. The method includes inserting fasteners into the fastener apertures in the caddy. The caddy is then coupled to a bone plate such that the fastener and the fastener apertures of the caddy are aligned with the fastener apertures in the bone plate. When the caddy is coupled to the bone plate, the clip portions are engaged to a lower surface of the bone plate. The method includes advancing the fasteners to secure the bone plate to the bone. The method includes removing the caddy from the bone plate by inserting an insertion tool into the inserter aperture in the caddy to deflect at least one of the clip portions.

Yet another aspect of the present invention is a surgical fastener caddy guide. The caddy guide includes a caddy body that has a top surface, a bottom surface, and at least two side surfaces. The caddy body also has at least one fastener aperture extending through the caddy body from the top surface of the caddy body to the bottom surface of the caddy body. The caddy body includes an inserter aperture. One side surface of the caddy body includes a first clip member with a tab on the lower surface of the first clip member. The fastener aperture(s) includes at least one vertically extending finger that is located between two vertically extending slots in a side wall of the fastener aperture.

### BRIEF DESCRIPTION OF THE DRAWINGS

Examples of the preferred embodiments are shown in the drawings. It should be appreciated, however, that the invention is not limited to the precise arrangements and instrumentally shown. In the drawings:

FIG. 1 is a bottom perspective view of two fastener caddies coupled to a bone plate;

FIG. 2 is a top perspective view of the bone plate and caddy assembly shown in FIG. 1;

FIG. 3 is a top view of the bone plate and caddy assembly shown in FIG. 1 with a single fastener;

FIG. 4 is a bottom view of the bone plate and caddy assembly shown in FIG. 3;

FIG. 5 is a front perspective view of a fastener;

FIG. 6 is a side perspective view of a fastener;

FIG. 7 is a top view of a fastener;

FIG. 8 is a bottom view of a fastener;

FIG. 9 is a top perspective view of the bone plate and caddy assembly shown in FIG. 2 with one of the two fastener caddies removed;

FIG. 10 is a bottom perspective view of the bone plate and caddy assembly shown in FIG. 9;

FIG. 11 is a top view of the bone plate and caddy assembly shown in FIG. 9;

FIG. 12 is a bottom view of the bone plate and caddy assembly shown in FIG. 9;

FIG. 13 is a front perspective view of another embodiment of a bone plate;

FIG. 14 is a top view of the bone plate shown in FIG. 13;

FIG. 15 is a bottom view of the bone plate shown in FIG. 13;

FIG. 16 is a front perspective view of another embodiment of a bone plate;

FIG. 17 is a bottom view of the bone plate shown in FIG. 16;

FIG. 18 is a top view of the bone plate shown in FIG. 16;

FIG. 19 is a front perspective view of another embodiment of the bone plate;

FIG. 20 is a top view of the bone plate shown in FIG. 19;

FIG. 21 is a bottom view of the bone plate shown in FIG. 19;

FIG. 22 is a front perspective view of another embodiment of a bone plate;

FIG. 23 is a top view of the bone plate shown in FIG. 22;

FIG. 24 is a bottom view of the bone plate shown in FIG. 22;

FIG. 25 is a side perspective view of one embodiment of a fastener caddy;

FIG. 26 is a top view of the fastener caddy shown in FIG. 25;

FIG. 27 is a bottom view of the fastener caddy shown in FIG. 25;

FIG. 28 is a side perspective view of another embodiment of a fastener caddy;

FIG. 29 is a top view of the fastener caddy shown in FIG. 28;

FIG. 30 is a bottom view of the fastener caddy shown in FIG. 28;

FIG. 31 is a perspective view of the bone plate shown in FIG. 22 utilizing the fastener caddies shown in FIG. 25;

FIG. 32 is a top perspective view of the assembly shown in FIG. 31;

FIG. 33 is a bottom view of the assembly shown in FIG. 31;

FIG. 34 is a front perspective view of the bone plate shown in FIG. 19 with the fastener caddy shown in FIG. 25;

FIG. 35 is a top view of the bone plate and fastener caddy assembly shown in FIG. 34;

FIG. 36 is a bottom view of the bone plate and fastener caddy assembly shown in FIG. 34;

FIG. 37 is a partial cross-sectional view of an insertion tool within a fastener aperture of the fastener caddy;

FIG. 38 is a partial front perspective view of an insertion tool being used to remove a fastener caddy from a bone plate;

FIG. 39 is a partial cross-sectional view of an insertion tool before it is inserted into the insertion tool aperture in a fastener caddy;

FIG. 40 is a side perspective view of the insertion tool with a handle; and

FIG. 41 is a side perspective view of the insertion tool with the handle removed.

### DETAILED DESCRIPTION

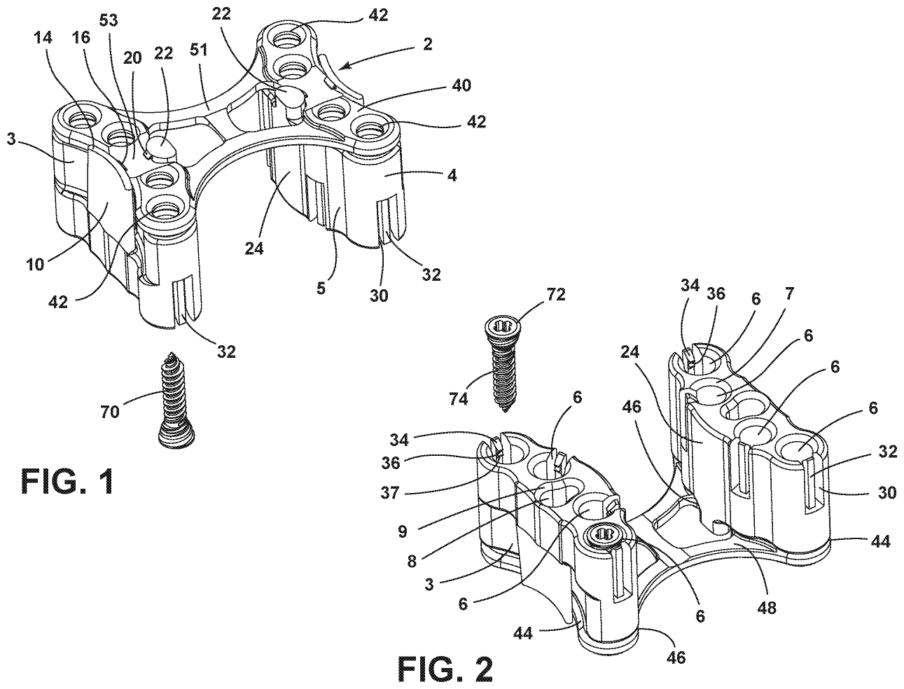

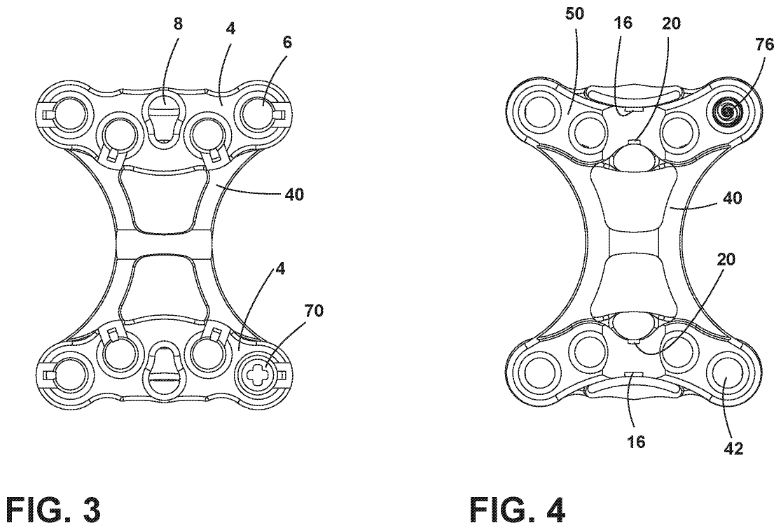

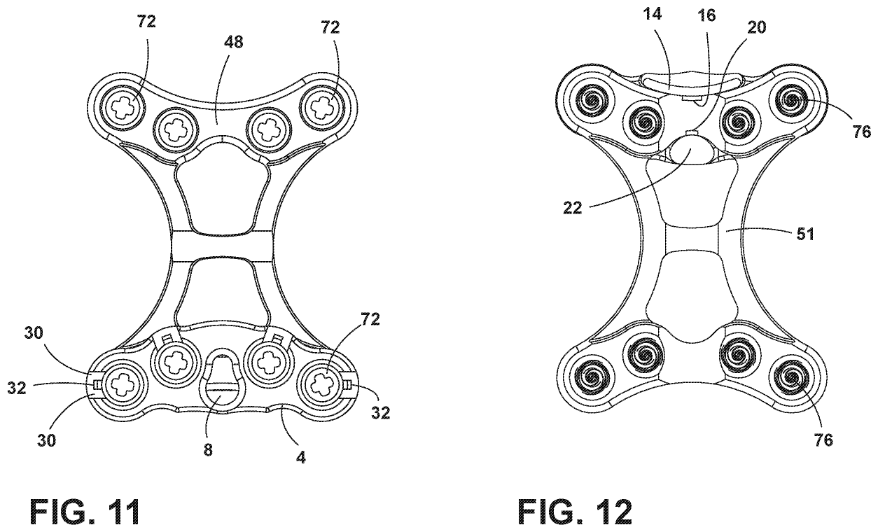

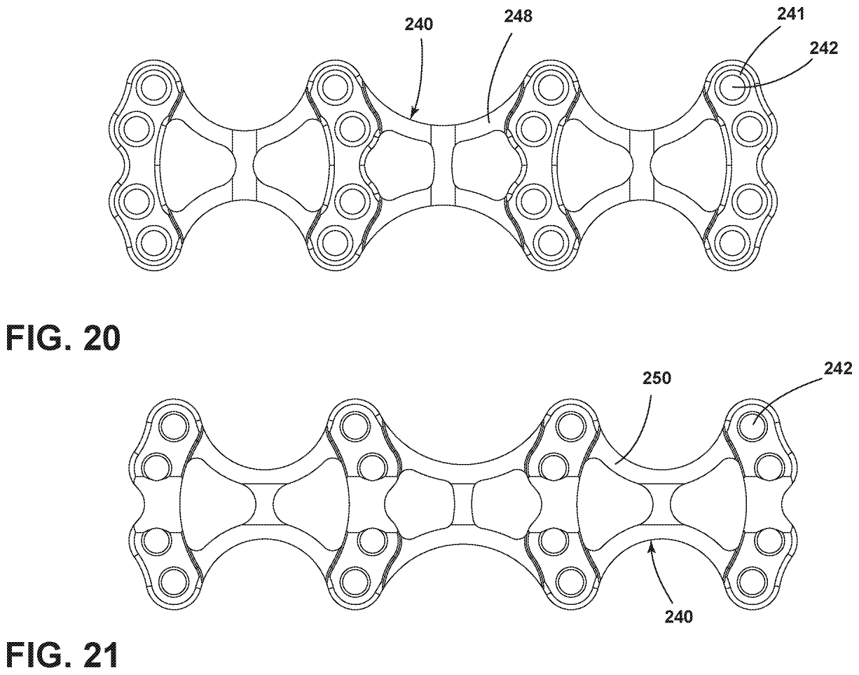

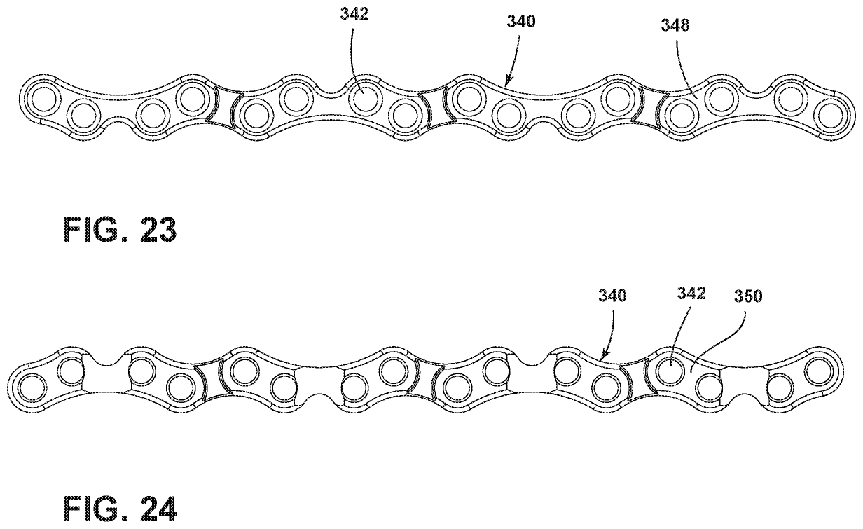

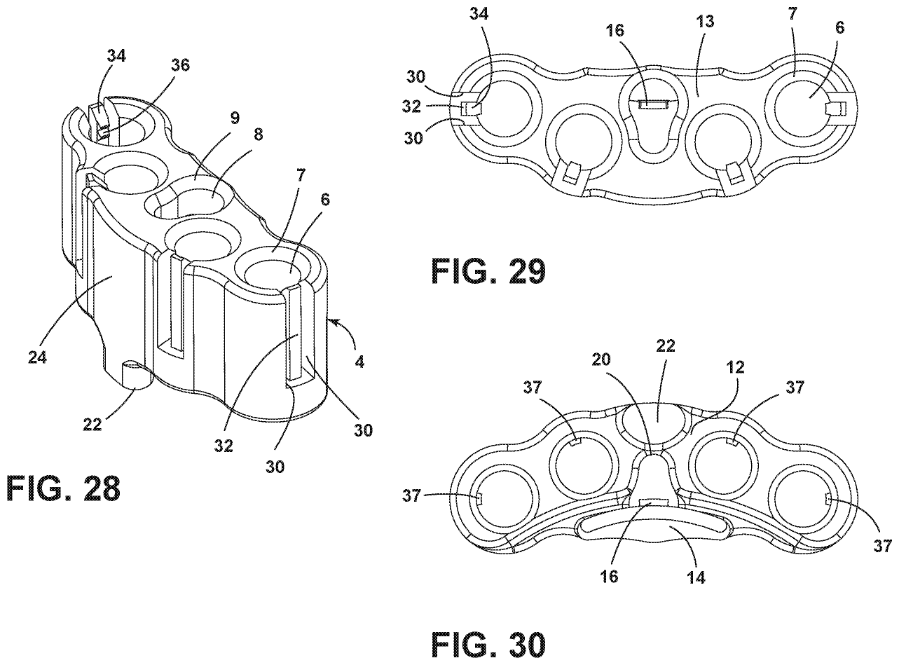

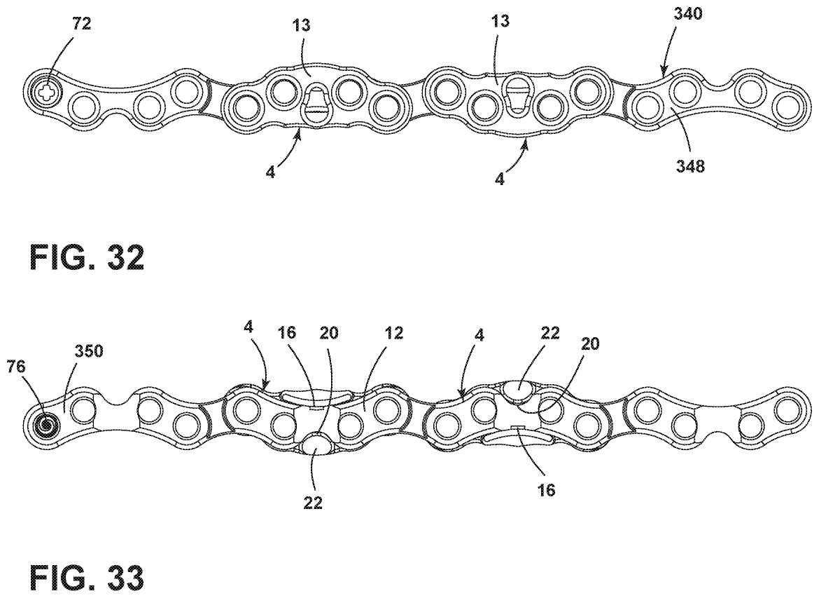

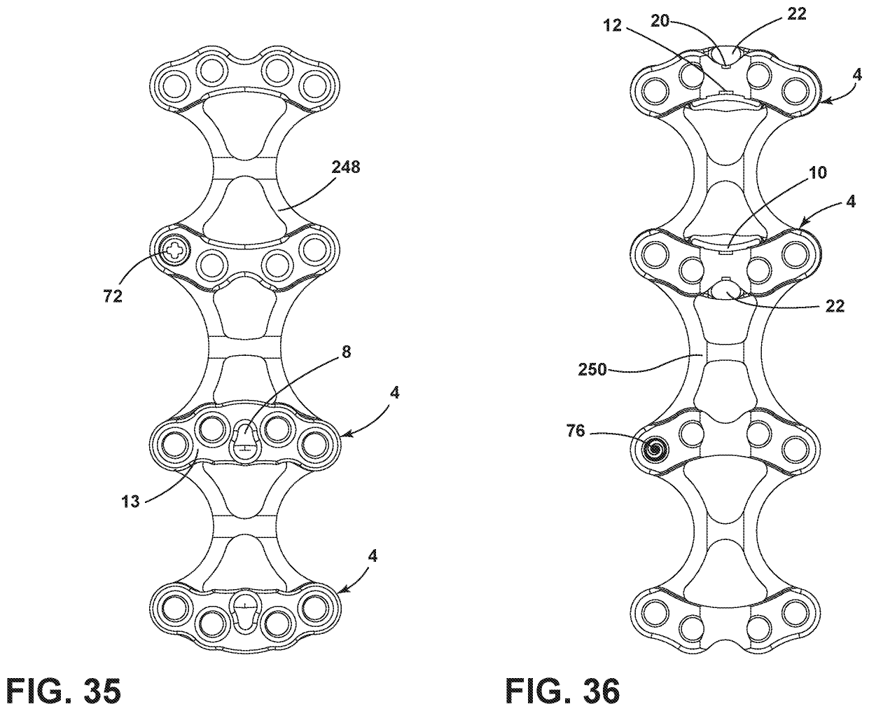

For purposes of description herein, the terms “upper,” “lower,” “right,” “left,” “rear,” “front,” “vertical,” “horizontal,” and derivatives thereof shall relate to the invention as oriented in FIGS. 1-41. However, it is to be understood that the invention may assume various alternative orientations and step sequences, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

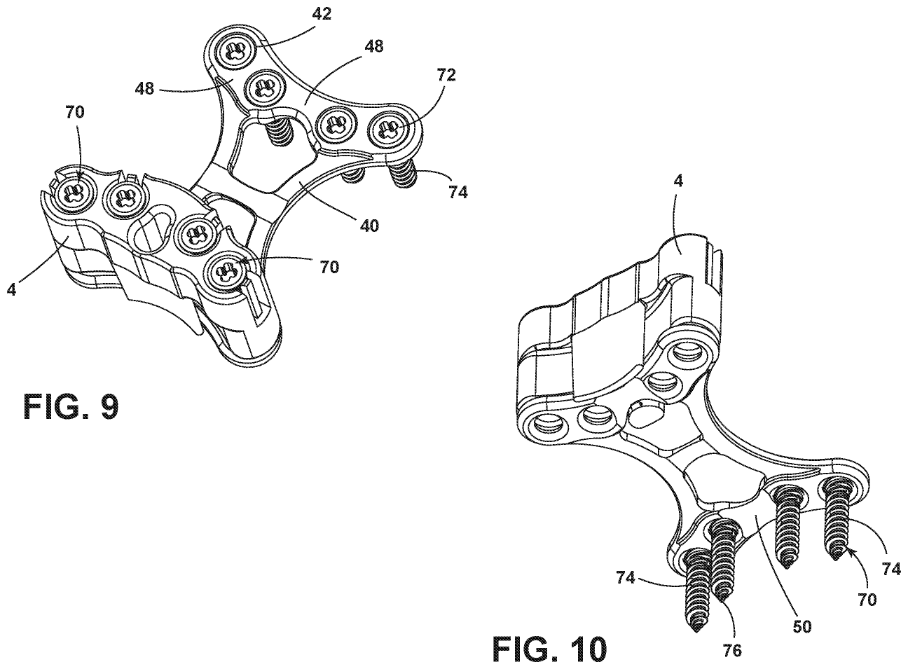

FIGS. 1-4 and 9-12 illustrate one embodiment of a fastener caddy that is useful for understanding the inventive concepts disclosed herein. The fastener caddy assembly 2 includes a fastener caddy 4 that is coupled to a bone plate 40. A number of fasteners 70 are preloaded into the fastener caddy 4, as illustrated in FIG. 9, and the fastener caddy 4 can be removed from the bone plate 40 once the fasteners 70 have been installed into the bone plate 40.

The fastener caddy 4 has a top surface 13 and a bottom surface 12. Fastener caddy 4 also has sides 3 and 5 that have the same general contour as the segment of the bone plate 40 to which it is coupled. The fastener caddy 4 also has a number of fastener apertures 6. The fastener apertures 6 can be unthreaded, partially threaded, or entirely threaded. In the illustrated embodiments, the fastener caddy 4 has four fastener apertures 6, although any number of fastener apertures can be used depending upon the size and purpose of the associated bone plate 40. The top surface of the fastener aperture 6 includes a tapered portion 7. The tapered portion 7 will generally engage a surface of the head 72 of a fastener 70 when the fasteners are preloaded into the fastener caddy 4. The fastener caddy 4 also has an insertion tool aperture 8 with a similarly tapered surface 9.

The fastener caddy 4 couples to the bone plate 40 by the engagement of tabs 16 and 20. Specifically, the fastener caddy 4 has a first clip 10 on side 3 with a lower surface 14 and a tab 16 extending therefrom. The opposite side 5 of the fastener caddy 4 has a second clip 24 with a bottom surface 22 and a tab 20. As illustrated in FIGS. 1 and 4, the tabs 16, 20 engage the bottom surface 50 of bone plate 40.

As illustrated in FIGS. 1-4, bone plate 40 has a number of fastener apertures 42 that extend from the top surface 48 to the bottom surface 50 of the bone plate 40. The bone plate 40, illustrated in FIGS. 1-4 and 9-12, has a first side 44 and a second side 46. The first side 44 is on the side 3 of fastener caddy 4, while the side 46 is on the side 5 of fastener caddy 4. The bone plate 40 illustrated in FIGS. 1-4 and 9-12 has two segments that are connected by a middle portion 51. Thus, the bone plate 40 has two segments that are spaced apart, which each segment having the first side 44 and the second side 46. As illustrated in these Figures, the fastener apertures 6 of fastener caddy 4 are in alignment with the fastener apertures 42 of bone plate 40 when fastener caddy 4 is coupled to the bone plate 40. As illustrated in FIG. 9, this permits the fasteners 70 to be preloaded into the fastener caddy 4 and situated above the fastener apertures 42 and bone plate 40 before the fasteners 70 are driven by the surgeon.

FIG. 9 illustrates one side of the bone plate 40 having the fasteners 70 already installed into the fastener aperture 42 of the bone plate 40, while the other side still has the fastener caddy 4 with the preloaded fasteners 70. The fastener caddy 4 is removed once the fasteners 70 have been installed into the bone plate 40 by the insertion of an insertion tool into insertion tool aperture 8. The tool has a section that presses one or both of the clip portions 10, 24 of the fastener caddy 4 to disengage the one or both of the tab members 16, 20 from the bottom surface 50 from the bone plate 40 thereby permitting the decoupling and removal of the fastener caddy 4 from bone plate 40.

The embodiment of the fastener caddy 4 illustrated in FIGS. 1-4 and 9-12 has at least one finger 32 located on each of the fastener apertures 6. The finger 32 is formed in a side wall of the fastener aperture 6 in between two vertical slots 30. The finger 32 includes tapered surface 34 that generally matches the tapered surface 7 of the fastener aperture 6. The finger 32 also includes a groove 36 located below the tapered surface 34 that is located between the tapered surface 34 and an extension 37. The groove 36 interfaces with one or more threads on the threaded shank 74 of the fastener 70, preventing the fastener 70 from falling out of the fastener aperture 6 in fastener caddy 4. The interface between the groove 36 and the threaded shank 74 allows for a variation in the length and diameter of the fastener 70 that is inserted into the fastener aperture 6 in fastener caddy 4. The shape of the groove 36 can match the thread pitch of the threaded shank 74 and lock the fastener 70 in place in the fastener aperture 6. When the pre-installed fasteners 70 are advanced toward the bone plate 40, the fingers 32 will flex and permit the fasteners 70 to be advanced toward and installed into the bone plate 40. While the illustrated embodiment, in FIGS. 1-4 and 9-12, show a single finger 32 with each fastener aperture 6, multiple fingers 32 can be used with any given fastener aperture 6.

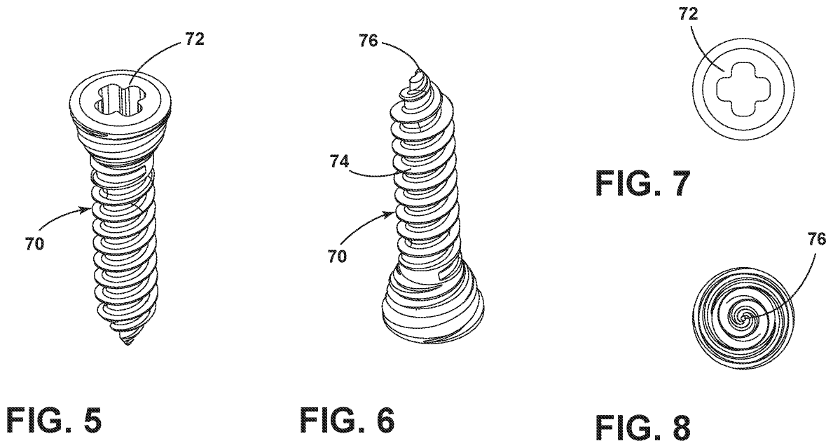

As illustrated in FIGS. 5-8, fastener 70 includes a head 72 with a shaped surface for being advanced by a driving bit. In the illustrated embodiments, the head 72 receives a Phillips driving bit. The threaded shank 74 of the fastener 70 terminates in a lower tip 76.

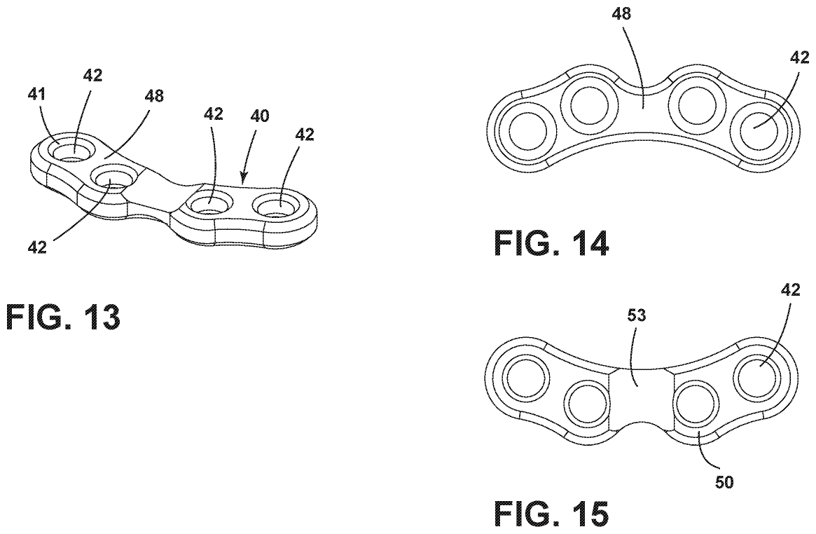

While FIGS. 1-4 and 9-12 illustrate a bone plate 40 with two segments that are connected by middle portion 51, FIGS. 13-15 illustrate a single segment of the same bone plate 40. Again, the bone plate 40 has fastener apertures 42 with a tapered surface 41 that will engage a portion of the head 72 of fastener 70 when the fasteners are fully installed into the bone plate 40. The bone plate 40 also has an top surface 48 and a bottom surface 50.

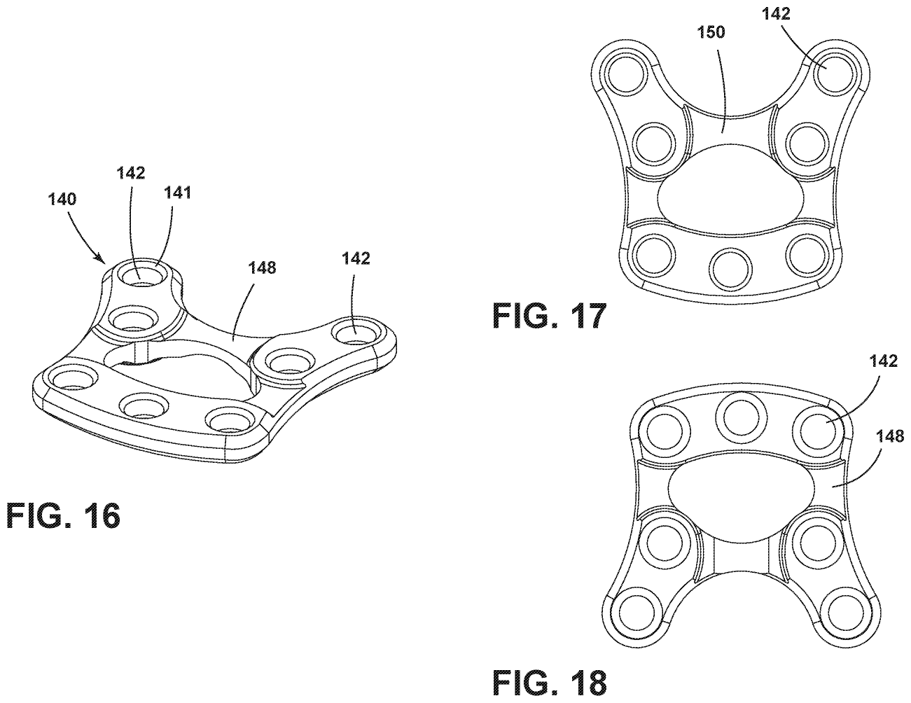

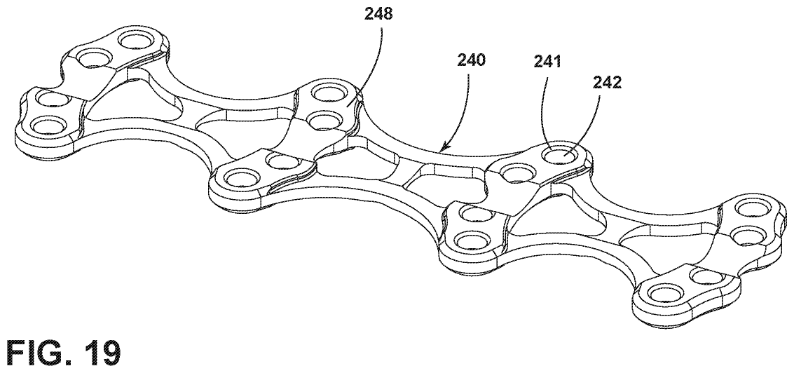

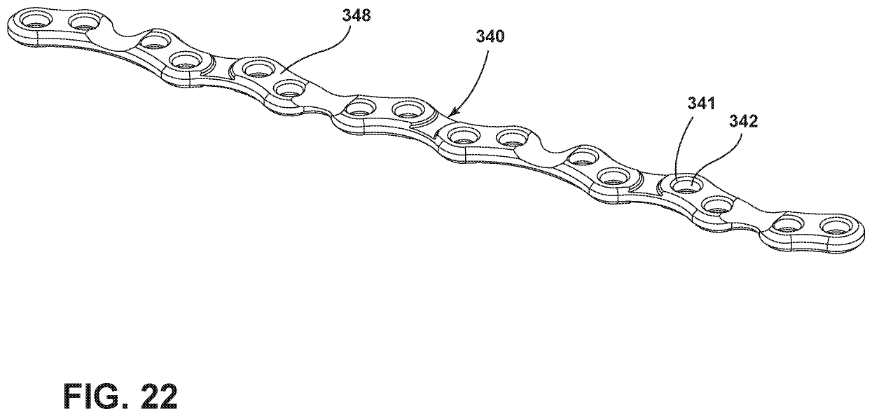

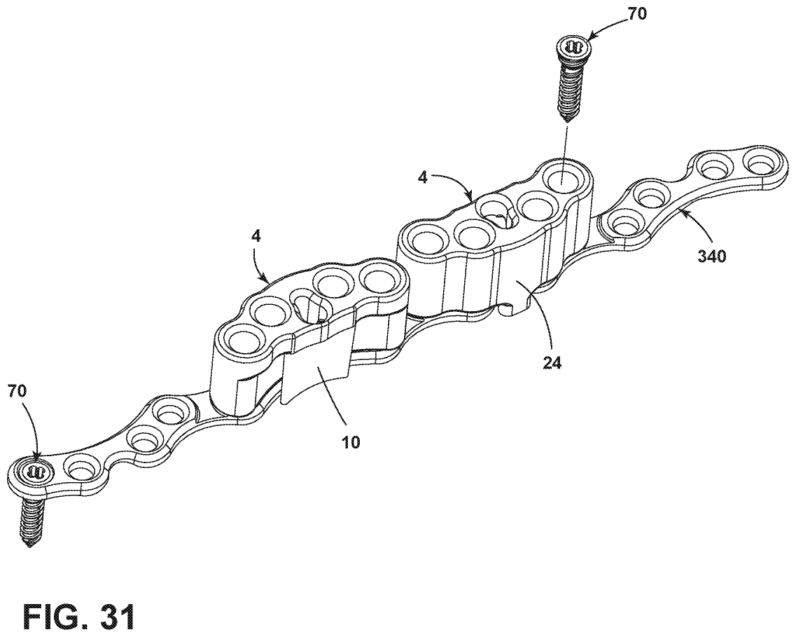

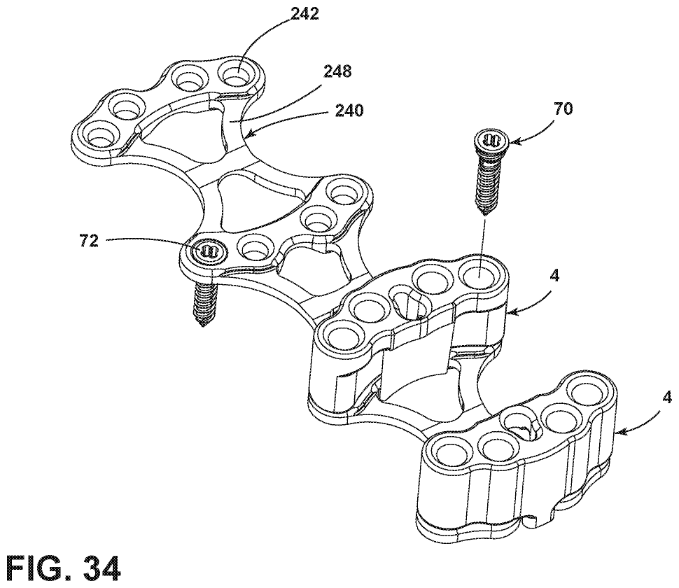

FIGS. 16-18 illustrate another embodiment of a bone plate 140. That bone plate 140 has a plurality of fasteners apertures 142 with a rim 141 that will engage a portion of the head 72 of fastener 70 when the fastener 70 is fully installed into the fastener aperture 142. The bone plate 140 has a top surface 148 and a bottom surface 150. FIGS. 19-21 illustrate another embodiment of a bone plate 240. This embodiment also includes a plurality of fastener openings 242 with a rim 241. The bone plate 240 has a top surface 248 and a bottom surface 250. FIGS. 22-24 illustrate another embodiment of a bone plate 340. That bone plate 340 includes a top surface 348, a bottom surface 350, and a number of fastener apertures 342 having a rim 341. FIGS. 31-33 illustrate the use of two fastener caddies 4 on segments of bone plate 340. Similarly, FIGS. 34-36 illustrate the use of multiple fastener caddies 4 on segments of bone plate 240. As can be seen from these embodiments, multiple fastener caddies 4 can be used with a given bone plate, and the fastener caddies 4 can be placed along the length, width, or other positions on the bone plate.

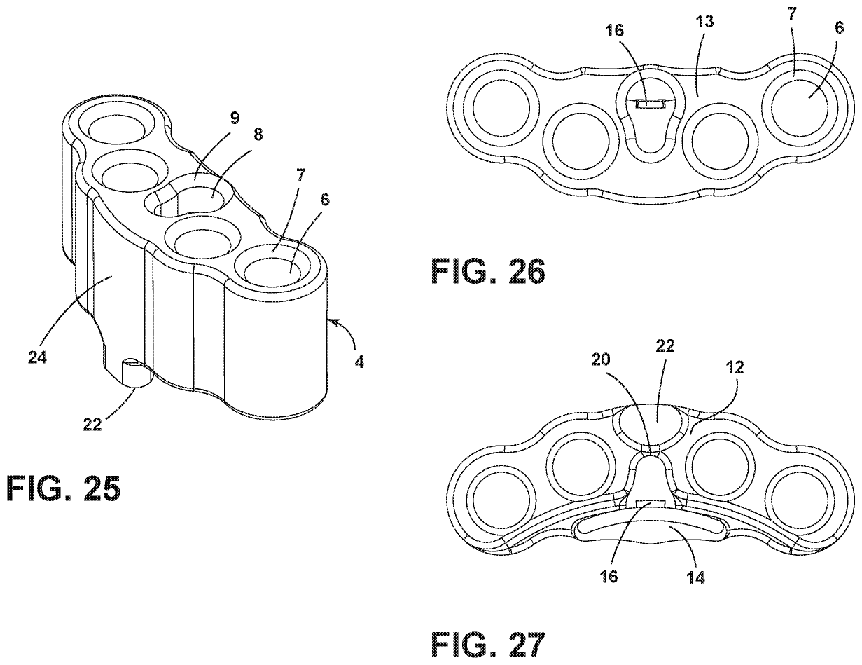

FIGS. 25-27 illustrate an alternative embodiment of the fastener caddy 4. The fastener caddy 4 in that embodiment does not utilize fingers 32.

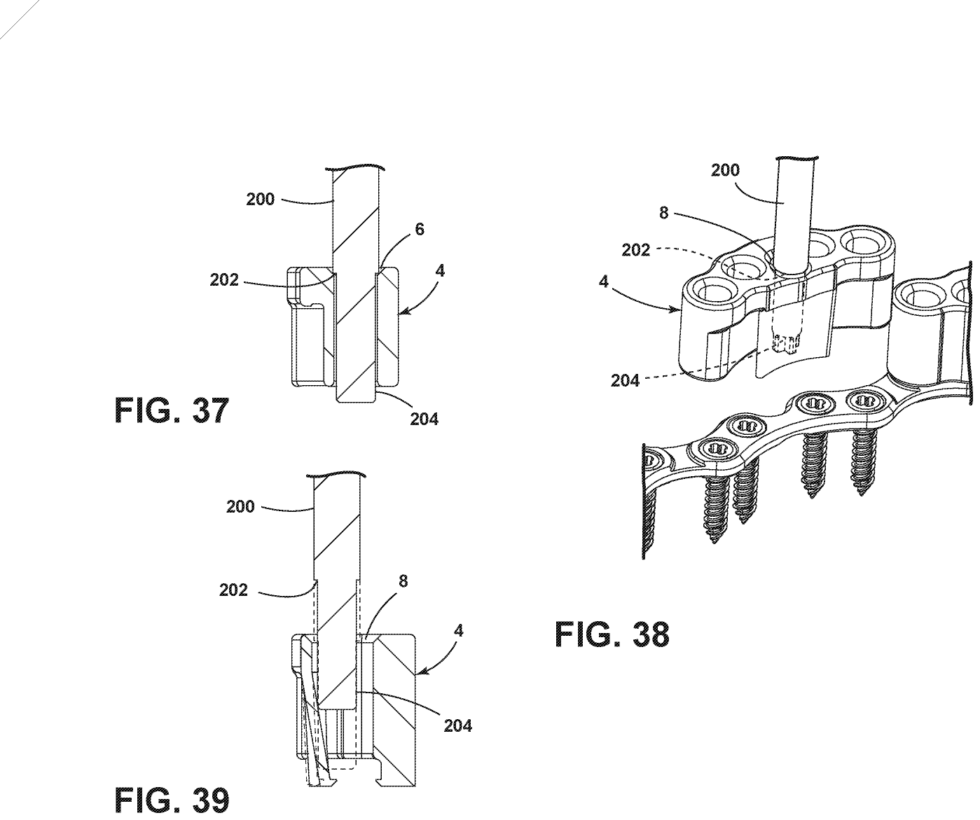

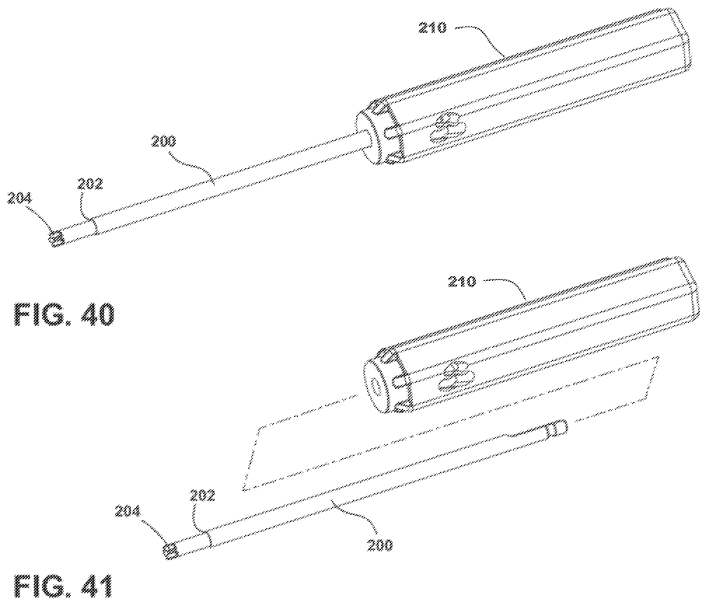

The insertion tool 200 is illustrated in FIGS. 40 and 42. The insertion tool 200 can be attached to a handle 210 for manual operation or to a powered device, such as a drill or driver. The insertion tool 200 has a head 204 and a shoulder 202. The head 204 can be shaped so that the insertion tool 200 can drive the fasteners 70 in fastener aperture 6, as illustrated in FIG. 37. Thus, in the illustrated embodiment, the head 204 of the insertion tool 200 has a Phillips head that drives head 72 of fastener 70. The shoulder 202 on insertion tool 200 is spaced to be at a height necessary to fully driver fastener 70 into fastener aperture 42 in bone plate 40. The shoulder 202 will engage tapered surface 7 of fastener aperture 6 when fully inserted into fastener aperture 6, i.e., after the fastener 70 has been fully driven. The shoulder 202 therefore prevents overtightening of the fastener 70 to bone plate 40 and also is of sufficient length to fully drive the fastener 70 into the fastener opening 42.

The insertion tool 200 is also used to remove the fastener caddy 4 from the bone plate 40, as illustrated in FIGS. 38-39. The insertion tool 200 will force at least one of the tabs 16, 20 to disengage from the bone plate 40. In the illustrated embodiment of FIG. 39, tab 16 on clip 10 is removed by the advancement of insertion tool 200 into insertion tool aperture 8. The shoulder 202 on insertion tool 200 will be stopped by tapered surface 9 of the insertion tool aperture 8 in the fastener caddy 4.

In use, the fastener caddy 4 has preloaded fasteners 70 in the fastener aperture 6 of the fastener caddy 4. The fastener caddy 4 is coupled to the bone plate 40 by the use of the tabs 16, 20 that engage the lower surface 50 of the bone plate 40. When the fastener caddy 4 is coupled to the bone plate 40, the fastener apertures in the fastener caddy 4 are aligned with the fastener apertures 42 of the bone plate 40. The contours of the sides 3, 5 of the fastener caddy 4 match the contours of the sides 44, 46 of the segment of the bone plate 40 to permit the alignment of the fastener aperture 6, 42. Once the bone plate 40 is aligned on the segment of bone during surgery, the surgeon advances the fasteners 70 through the fastener apertures 6 in fastener caddy 4 until the fasteners 70 are fully installed in the fastener apertures 42 of the bone plate 40. Once the preloaded fasteners 70 in the fastener caddy 4 are advanced into the bone plate 40, the fastener caddy 4 is removed by use of an insertion tool 200 that is inserted into the insertion tool aperture 8. The insertion tool 200 presses against one or both of the clips 10, 24 of the fastener caddy 4 to disengage one or both of tabs 16, 20 from the bottom surface 50 of the bone plate 40 permitting the fastener caddy 4 to be removed laterally and/or upwards.

In the illustrated examples, the lower surface 14 of clip 10 and the bottom surface 22 of clip 24 do not extend beyond the bottom surface 50 of bone plate 40. That is because bone plate 40 has an indented surface 53 in the area of tabs 16, 20. This permits the portions of the bone plate 40 to rest directly against the bone and to permit removal of the fastener caddy 4 from bone plate 40 once all fasteners 70 have been installed.

The fastener caddy 4 can be made of any suitable material that permits one or more of the clips 10, 24 to be deflected by the insertion tool. The fastener caddy 4 can be made from a polymeric material, metal, or a combination thereof. For example, the fastener caddy 4 can be made by injection molding a polymeric material, such as plastic and/or resin. The fastener caddy 4 can also be machined or stamped from a polymeric or metal material. The fastener caddy 4 can also be made from additive manufacturing or 3D printing. The flexibility of the material used in the fastener caddy 4 permits the installation of the fasteners 70 into the bone plate 40 by permitting the tapered portion of the head 72 to deflect the sides of the fastener opening 6 as the fastener 70 is advanced toward the bone plate 40.

The bone plate 40 can also be made of a biocompatible polymeric and/or metal material that is suitable for installation to the bone. Similarly, the fasteners 70 can also be made of a biocompatible polymeric and/or metal material.

The shape of the fastener caddy 4 can vary depending upon the shape of the bone plate 40. The shape of the fastener caddy 4 will require the alignment of the fastener aperture 6 in the fastener caddy 4 with the fastener aperture 42 in the bone plate 40. The shape of the fastener caddy 4 will also require at least one clip with a tab that can engage a lower surface of the bone plate 40.

While the illustrated embodiments show the fastener caddy 4 being coupled to bone plate 40 by the use of a clipping mechanism, the fastener caddy 4 could be coupled to the bone plate 40 by other mechanisms. For example, the fastener caddy 4 could be coupled to the bone plate 40 by use of one or more removable fasteners, such as screws, adhesive tape, etc. The fastener caddy 4 could also be coupled to the bone plate 40 by use of a releasable liner, such as tape, adhesive, and hook-and-loop fasteners.

The fastener caddy 4, fasteners 70, and bone plate 40 can be packaged together. Thus, a single, sterilized package could contain the fastener caddy 4 coupled to the bone plate 40 with the fasteners 70 already installed in the fastener apertures 6 of the fastener caddy 4. Alternatively, the fastener caddy 4 could be packaged with the fasteners 70 installed or uninstalled. The fastener caddy 4 can be disposable such that once the fasteners 70 are installed into the bone plate 40 and the fastener caddy 4 is decoupled from the bone plate 40, the fastener caddy 4 is disposed. Alternatively, the fastener caddy 4 can be reused if it is sterilized again.

In the foregoing description, it will be readily appreciated by those skilled in the art that modifications may be made to the invention without departing from the concepts disclosed herein. Such modifications are to be considered as included in the following claims, unless these claims by their language expressly state otherwise.

It will be understood by one having ordinary skill in the art that construction of the present disclosure and other components is not limited to any specific material. Other exemplary embodiments of the disclosure disclosed herein may be formed from a wide variety of materials, unless described otherwise herein.

For purposes of this disclosure, the term “coupled” or “operably coupled” (in all of its forms, couple, coupling, coupled, etc.) generally means the joining of two components (electrical or mechanical) directly or indirectly to one another. Such joining may be stationary in nature or movable in nature. Such joining may be achieved with the two components (electrical or mechanical) and any additional intermediate members being integrally formed as a single unitary body with one another or with the two components. Such joining may be permanent in nature or may be removable or releasable in nature unless otherwise stated. In addition, while certain embodiments have shown threaded connections, the threaded connections could include tape or other sealing material in the threaded connection. In addition, the threaded connection could be replaced by other suitable connections or couplings, such as compression couplings or other couplings.

For purposes of this disclosure, the term “connected” or “operably connected” (in all of its forms, connect, connecting, connected, etc.) generally means that one component functions with respect to another component, even if there are other components located between the first and second component, and the term “operable” defines a functional relationship between components.

It is also important to note that the construction and arrangement of the elements of the present disclosure as shown in the exemplary embodiments is illustrative only. Although only a few embodiments of the present innovations have been described in detail in this disclosure, those skilled in the art who review this disclosure will readily appreciate that, unless otherwise described, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. It should be noted that the elements and/or assemblies of the system may be constructed from any of a wide variety of materials that provide sufficient strength or durability, in any of a wide variety of colors, textures, and combinations. Accordingly, all such modifications are intended to be included within the scope of the present innovations. Other substitutions, modifications, changes, and omissions may be made in the design, operating positions, and arrangement of the desired and other exemplary embodiments without departing from the spirit of the present innovations.

It will be understood that any described processes or steps within described processes may be combined with other disclosed processes or steps to form structures within the scope of the present disclosure. The exemplary structures and processes disclosed herein are for illustrative purposes and are not to be construed as limiting.

It is also to be understood that variations and modifications can be made on the aforementioned structures and methods without departing from the concepts of the present invention, and further it is to be understood that such concepts are intended to be covered by the following claims unless these claims by their language expressly state otherwise.

1. A surgical plate and caddy assembly, comprising:
a bone plate having a top surface, a bottom surface, and a plurality of fastener apertures;
a caddy coupled to said bone plate, said caddy having a top surface, a bottom surface, and at least two side surface, at least one removal aperture, and a plurality of fastener apertures that are aligned with said plurality of fastener apertures in said bone plate;
wherein the caddy includes a clip portion having a tab that contacts said bone plate in order to couple the caddy to the bone plate;
wherein the clip portion deflects to remove said tab from contact with said bone plate in order to remove the caddy from the bone plate; and
wherein the at least one removal aperture provides an access to deflect the clip portion from contact with the bone plate; and
a plurality of fasteners inserted into and held in said plurality of fastener apertures in said caddy by a flexible interface that deflects when the fasteners are advanced through the caddy fastener apertures into the aligned fastener apertures of the bone plate.

2. The surgical plate and caddy assembly of claim 1, wherein:
the caddy includes a further clip portion having a tab that contacts said bone plate in order to couple the caddy to the bone plate and that deflects to remove the caddy from the bone plate, wherein the at least one removal aperture provides access to both clip portions to deflect both clip portions from contact with the bone plate.

3. The surgical plate and caddy assembly of claim 1, wherein:
each fastener includes a head, and wherein each of said plurality of fastener apertures in said caddy includes a rim, and wherein each rim engages a surface on the head of a fastener held in the fastener aperture.

4. The surgical plate and caddy assembly of claim 1, wherein:
each of said plurality of fastener apertures in said caddy includes a sidewall, and wherein each sidewall includes at least one slot.

5. The surgical plate and caddy assembly of claim 4, further comprising a flexible finger portion in a slot of a fastener aperture.

6. The surgical plate and caddy assembly of claim 5, wherein said flexible finger portion includes a surface that tapers from the top surface of the flexible finger portion toward the center of said fastener aperture in said caddy, and a groove located on the surface of the flexible finger that is oriented toward the center of said fastener aperture.

7. A method of installing fasteners into a bone plate, comprising:
providing a caddy to have a top surface, a bottom surface, at least one deflectable clip portion having a tab, at least one removal aperture providing an access to the clip portion, and a plurality of fastener apertures;
inserting fasteners into said fastener apertures in said caddy, wherein the fasteners are held in the caddy fastener apertures by a flexible interface that deflects when the fastener apertures are advanced through the caddy fastener apertures;
using the at least one deflectable clip portion to removably couple said caddy to a bone plate comprising a plurality of fastener apertures such that the fastener apertures of said caddy are aligned with said fastener apertures in said bone plate;
while the caddy is coupled to the bone plate, advancing said fasteners through the caddy fastener apertures and the bone plate fastener apertures to secure said bone plate to a bone; and
using the at least one removal aperture to access and deflect the clip portion to remove said caddy from said bone plate.

8. (canceled)

9. (canceled)

10. The method of installing fasteners into a bone plate of claim 7, wherein each of said plurality of fastener apertures in said caddy includes at least one deflectable finger located in a slot in a side wall of said fastener aperture.

11. The method of installing fasteners into a bone plate of claim 10, wherein said vertically extending fingers deflect as said fasteners are advanced toward said bone plate.

12. The method of installing fasteners into a bone plate of claim 7, wherein multiple caddies are coupled to a single bone plate.

13. The method of installing fasteners into a bone plate of claim 7, wherein each of said plurality of fastener apertures in said caddy includes at least one surface to stop the advancement of a fastener to prevent the fastener from being over-fastened into said bone plate.

14. The method of installing fasteners into a bone plate of claim 7, wherein an insertion tool is inserted through the at least one insertion aperture to deflect the at least one clip portion.

15. A surgical fastener caddy guide that is used with a bone plate, comprising:
a caddy body having a top surface, a bottom surface, and at least two side surfaces;
at least one fastener aperture having a side wall including a slot;
wherein said caddy body includes a first, deflectable clip member with a tab that engages the bone plate to couple the caddy guide to the bone plate and that deflects to remove the caddy guide from the bone plate;
at least one clip member aperture in said caddy body providing an access to deflect the first clip member; and
a preloaded fastener held in the at least one fastener aperture; and
wherein said at least one fastener aperture includes at least one deflectable finger that is located in the slot of the side wall, said deflectable finger providing a flexible interface that holds the preloaded fastener in the at least one fastener aperture and that deflects to allow the preloaded fastener to advance through the at least one fastener aperture.

16. The surgical fastener caddy guide of claim 15, wherein said caddy comprises a polymeric material.

17. The surgical fastener caddy guide of claim 15, including:
a second deflectable clip member on the caddy guide, said second deflectable clip member having a tab on the lower surface of the second clip member that engages the bone plate to couple the caddy guide to the bone plate and that deflects to remove the caddy guide from the bone plate; and wherein the at least one clip member aperture provides an access to the second deflectable clip member.

18. The surgical fastener caddy guide of claim 15, wherein said fastener aperture includes at least one internal surface for stopping the advancement of a fastener through said fastener aperture.

19. The surgical fastener caddy guide of claim 15, wherein said clip member aperture extends through said caddy body and is positioned above said tab of said first clip member.

20. The surgical caddy fastener guide of claim 16, wherein said polymeric material is an elastomeric polymer material.

21. A surgical bone plate and preloaded fastener caddy combination, comprising:
a) a surgical bone plate comprising one or a multiple of bone plate fastener apertures;
b) a fastener caddy that is configured to be removably coupled to the surgical bone plate, wherein the fastener caddy comprises at least one caddy fastener aperture that is alignable with a bone plate fastener aperture when the fastener caddy is removably coupled to the surgical bone plate, and wherein the alignable caddy fastener aperture includes one or more flexible engagement elements that are configured to provide a flexible interface that holds a fastener in the alignable caddy fastener aperture and that deflects as the fastener held in the alignable caddy fastener aperture is advanced through the caddy fastener aperture; and
c) a preloaded fastener that is held in the alignable caddy fastener aperture by the flexible interface.

22. The combination of claim 21, wherein the one or more flexible engagement elements comprise at least one flexible finger.

23. The combination of claim 21, wherein the one or more flexible engagement elements comprise at least one vertically extending, flexible finger.

24. The combination of claim 23, wherein the alignable caddy fastener aperture comprises a slot in a side, and wherein a flexible finger is positioned in the slot.

25. The combination of claim 21, wherein the one or more flexible engagement elements comprise a side of the alignable caddy fastener aperture.

26. The combination of claim 21, wherein the one or more flexible engagement elements comprise a flexible material.

27. The combination of claim 21, wherein the surgical bone plate comprises a middle portion and first and second, spaced apart segments coupled to the middle portion, wherein each segment includes a surgical bone plate fastener aperture.

28. The combination of claim 27, wherein the middle portion of the surgical bone plate has a reduced thickness.

29. The combination of claim 27, wherein the middle portion of the surgical bone plate has an indented surface, and wherein the fastener caddy is configured to couple to the indented surface.

30. The combination of claim 21, wherein the combination includes a multiple of the fastener caddies, wherein each fastener caddy includes at least one preloaded fastener and wherein the surgical bone plate and the multiple of fastener caddies are configured so that the multiple of fastener caddies can be removably coupled to the surgical bone plate.

31. The combination of claim 21, wherein the fastener caddy comprises a flexible tab that engages the surgical bone plate and that deflects to disengage from the surgical bone plate.

32. The combination of claim 21, wherein the fastener caddy includes a plurality of said fastener apertures, and wherein a plurality of pre-loaded fasteners are held in the plurality of caddy fastener apertures.

33. The combination of claim 21, wherein the caddy aperture extends through the fastener caddy from a top caddy surface to a bottom caddy surface.

34. The combination of claim 21, wherein the caddy fastener aperture is unthreaded

35. The combination of claim 21, wherein the preloaded fastener comprises a head and a threaded shank, and wherein the flexible interface engages the fastener head.

36. The combination of claim 35, wherein the fastener head is tapered and threaded.

37. The combination of claim 36, wherein the one or more flexible engagement elements comprise a side of the caddy fastener aperture, and wherein the tapered, threaded head of the preloaded fastener deflects the side as the fastener is advanced through the alignable caddy fastener aperture.

38. A surgical bone plate and caddy assembly, comprising:
a) a surgical bone plate comprising a plurality of bone plate fastener apertures;
b) a fastener caddy that is removably coupled to the surgical bone plate, wherein the fastener caddy comprises at least one fastener aperture that is aligned with a bone plate fastener aperture, and wherein the aligned caddy fastener aperture includes one or more flexible engagement elements that are configured to provide a flexible interface that holds a fastener in the alignable caddy fastener aperture and that deflects as the fastener held in the alignable caddy fastener aperture is advanced through the caddy fastener aperture; and
c) a preloaded fastener that is held in the aligned caddy fastener aperture by the flexible interface while the fastener caddy is removably coupled to the surgical bone plate.

39. A method of installing a fastener into a surgical bone plate, comprising the steps of:
a) providing a fastener caddy comprising at least one caddy fastener aperture, wherein the caddy fastener aperture comprises one or more flexible engagement elements that are configured to provide a flexible interface that holds a fastener in the alignable caddy fastener aperture and that deflects as the fastener held in the alignable caddy fastener aperture is advanced through the caddy fastener aperture;
b) providing a preloaded fastener held in the caddy fastener aperture by the flexible interface;
c) providing an assembly in which the fastener caddy is removably coupled to the surgical bone plate while holding the preloaded fastener, wherein the surgical bone plate comprises a bone plate fastener aperture that is aligned with the caddy fastener aperture holding the preloaded fastener; and
d) advancing the preloaded fastener through the fastener caddy aperture and into the aligned bone plate aperture, wherein the flexible interface deflects to permit the preloaded fastener to be advanced through the caddy fastener aperture and into the aligned bone plate fastener aperture.

40. A surgical bone plate and preloaded fastener caddy combination, comprising:
a) a plurality of different surgical bone plates, wherein each of the different surgical bone plates has a plurality of fastener apertures, and wherein the different surgical bone plates have a common hole pattern;
b) a preloaded fastener caddy that is configured to be removably coupled to each of the surgical bone plates, wherein the fastener caddy comprises a plurality of caddy fastener apertures having a hole pattern that is the same as the common hole pattern of the different surgical bone plate fastener apertures such that the caddy fastener apertures align with the fastener apertures of each of the different surgical bone plates when the fastener caddy is removably coupled to each of the different surgical bone plates, and wherein each caddy fastener aperture includes one or more flexible engagement elements that are configured to provide a flexible interface that holds a fastener in the caddy fastener aperture and that deflects as the fastener held in the caddy fastener aperture is advanced through the caddy fastener aperture; and
c) at least one preloaded fastener that is held in a caddy fastener aperture by the flexible interface.

41. A method of making a surgical bone plate and fastener caddy combination, comprising the steps of:
a) providing a surgical bone plate comprising one or a multiple of bone plate fastener apertures;
b) providing a fastener caddy that is configured to be removably coupled to the surgical bone plate, wherein the fastener caddy comprises at least one caddy fastener aperture that is alignable with a bone plate fastener aperture when the fastener caddy is removably coupled to the surgical bone plate, and wherein the alignable caddy fastener aperture includes one or more flexible engagement elements that are configured to provide a flexible interface that holds a fastener in the alignable caddy fastener aperture and that deflects as the fastener held in the alignable caddy fastener aperture is advanced through the caddy fastener aperture; and
c) causing a fastener to be held in the alignable caddy fastener aperture by the flexible interface.
